(19) United States
(12) Patent Application Publication
(10) Pub. No.: US 2025/0375209 A1
(43) Pub. Date: Dec. 11, 2025

(54) ASPIRATION CATHETER WITH SHAPED DISTAL TIP

(71) Applicant: BOSTON SCIENTIFIC SCIMED, INC., Maple Grove, MN (US)

(72) Inventors: Robert D. Cooper, Andover, MN (US); Anthony Frank Tassoni, JR., Andover, MN (US); Alyssa Madej, Minnetonka, MN (US); Ryan Lee Reifsteck, Lakeville, MN (US); Jordon Lewis, Ramsey, MN (US)

(73) Assignee: BOSTON SCIENTIFIC SCIMED, INC., Maple Grove, MN (US)

(21) Appl. No.: 19/228,064

(22) Filed: Jun. 4, 2025

Related U.S. Application Data
(60) Provisional application No. 63/656,754, filed on Jun. 6, 2024.

(51) Int. Cl. A61B 17/22; A61M 25/00

(52) U.S. Cl. CPC A61B 17/22; A61M 25/0026; A61M 25/0068; A61B 2017/22079; A61B 2217/005; A61M 2025/0039

(57) ABSTRACT

An aspiration catheter for use in a thrombectomy procedure is disclosed. The aspiration catheter also includes distal tip member having a length and a lumen extending therein, wherein the distal tip member is coupled to the distal end region of the catheter shaft. A guidewire shaft extends within at least a portion of the lumen of the catheter shaft and the lumen of the distal tip member. A first portion of a distal facing outer surface of the distal tip member lies in a first plane, and a second portion of the distal facing outer surface of the tip member lies in a second plane, different from the first plane.

### CROSS REFERENCE TO RELATED APPLICATIONS

The present application claims the benefit of U.S. Provisional Patent Application Ser. No. 63/656,754, filed Jun. 6, 2024, the disclosure of which is incorporated herein by reference.

### TECHNICAL FILED

The disclosure is directed to aspiration systems. More particularly, the disclosure is directed to an aspiration catheter having a shaped distal tip member coupled to the aspiration catheter distal end.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be aspirated from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices. An example aspiration catheter includes a catheter shaft having a proximal end region, a distal end region and a lumen extending therein. The example aspiration catheter may also include a tip member having a length, a central longitudinal axis, a lumen extending therein and a distal opening. The tip member may be coupled to the distal end region of the catheter shaft. An example aspiration catheter may further include a guidewire shaft having a proximal end region and a distal end region. The guidewire shaft may extend within at least a portion of the lumen of the catheter shaft and the lumen of the tip member. The tip member may include a distal facing outer surface surrounding the opening of the tip member, whereby a first portion of the distal facing outer surface of the tip member may lie in a first plane, and a second portion of the distal facing outer surface of the tip member may lie in a second plane, different from the first plane.

Alternatively or additionally to any of the examples above, the first plane may be substantially orthogonal to the second plane.

Alternatively or additionally to any of the examples above, the second plane may intersect the first plane at a point distal of the proximal edge of the distal opening of the tip member.

Alternatively or additionally to any of the examples above, the second plane may intersect the first plane at an angle ranging between 15 and 60 degrees with respect to the first plane.

Alternatively or additionally to any of the examples above, the first plane may intersect the central longitudinal axis of the tip member at an angle of 90 degrees with respect to the central longitudinal axis of the tip member.

Alternatively or additionally to any of the examples above, a guidewire lumen may be disposed concentrically within the tip member and may accept passage of the guidewire shaft.

Alternatively or additionally to any of the examples above, the catheter shaft may further include a catheter shaft liner.

Alternatively or additionally to any of the examples above, a radiopaque marker may be disposed about the catheter shaft and near the tip member.

Alternatively or additionally to any of the examples above, the catheter shaft may further include a plurality of jet orifices.

Alternatively or additionally to any of the examples above, the catheter shaft may further include a grouping of at least two substantially adjacent jet orifices near the distal end of the catheter shaft and near the tip member.

Alternatively or additionally to any of the examples above, the catheter shaft may further include a saddle region, the saddle region formed of stainless steel and welded to the catheter shaft.

Another example aspiration catheter includes a catheter shaft having a proximal end region, a distal end region, and a lumen extending therein. In this and other examples, the aspiration catheter may further include a tip member having a length, a central longitudinal axis, a lumen extending therein and a distal opening. The tip member may be coupled to the distal end region of the catheter shaft. The aspiration catheter of this and other examples may further include a guidewire shaft having a proximal end region and a distal end region. The guidewire shaft may extend within at least a portion of the lumen of the catheter shaft and the lumen of the tip member. The tip member may include a distal facing outer surface surrounding the distal opening of the tip member, whereby the distal facing outer surface of the tip member extends along a convex path that runs transverse to the central longitudinal axis of the tip member. The aspiration catheter of this and other examples may further include a plurality of jet orifices.

Alternatively or additionally to any of the examples above, a guidewire lumen may be disposed concentrically within the tip member and may accept passage of the guidewire shaft.

Alternatively or additionally to any of the examples above, the convex path of the distal facing outer surface of the tip member may terminate in a vertex proximal to the distalmost end of the tip member.

Another example aspiration catheter includes a thrombectomy catheter including a catheter shaft having a proximal end region, a distal end region, a lumen extending therein, and a central longitudinal axis. The thrombectomy catheter of this and other examples may further include a tip member having a length, a lumen extending therein and a distal opening. The tip member may be coupled to the distal end region of the catheter shaft. The tip member may further include a distal facing outer surface surrounding the distal opening of the tip member, whereby at least a portion of the distal facing outer surface of the tip member follows a convex path comprising at least three inflection points.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an example thrombectomy system;

FIG. 2 is a partially exploded perspective view of a portion of the thrombectomy system of FIG. 1;

FIG. 3 is a longitudinal cross-sectional view of a distal end region of an example thrombectomy catheter;

FIG. 4 is a side view of an example catheter distal end region contemplated by the present disclosure.

FIG. 5 is a distal end view of the tip member of FIG. 4.

FIG. 6 is a side view of an example catheter distal end region contemplated by the present disclosure;

FIG. 7 is a distal end view of the tip member of FIG. 6.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure. The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters, aspiration catheters and systems may be used to remove thrombus, plaques, lesions, clots, etc. from veins or arteries. Some thrombectomy catheters and aspiration catheters may utilize high velocity saline jets in a series to entrain fluid or clot material into and through the shaft of the catheter. Other thrombectomy systems may utilize one or more pressurized saline jets which travel backwards to create a low-pressure zone and a vacuum effect, whereby the vacuum pulls clot material into and through the distal tip and shaft of the catheter. However, prolonged operation of a thrombectomy system may create increased forces placed on the distal tip of the thrombectomy catheter. Accordingly, it may be desirable to design a thrombectomy catheter and/or aspiration catheter which includes a distal tip member coupled near or coupled to the catheter distal end and configured to resist increased forces placed thereon. Thrombectomy systems which include a thrombectomy catheter having a strengthened distal tip bond configured to resist increased forces placed thereon are disclosed herein.

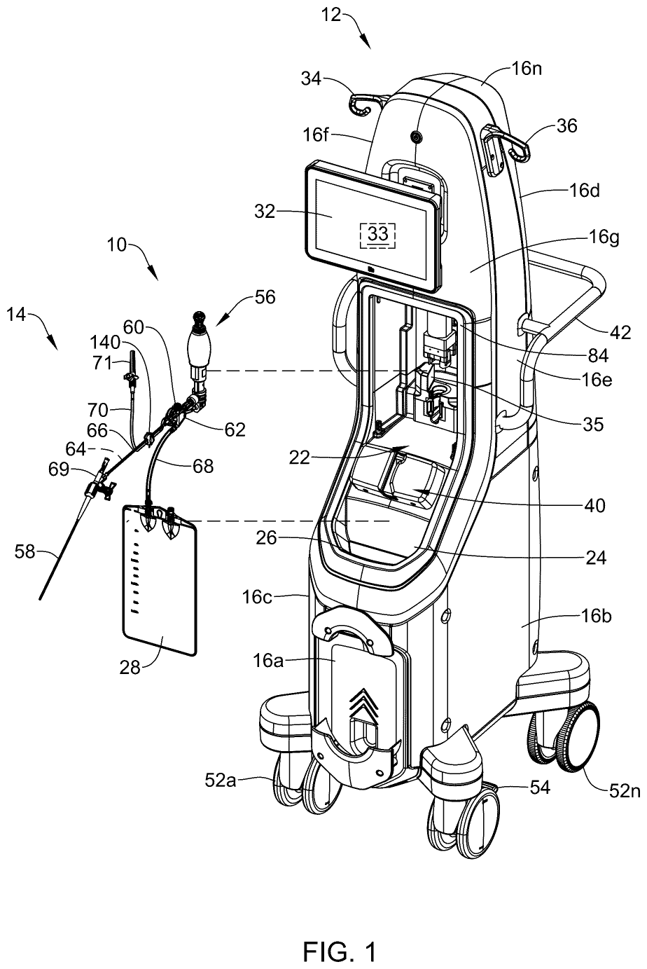

FIG. 1 is a perspective view of an illustrative thrombectomy system 10. The thrombectomy system 10 may include a control console or drive unit 12 and a pump/catheter assembly 14. In some instances, the pump/catheter assembly 14 may be a single use device in which a new pump/catheter assembly 14 may be used with the drive unit 12 for each medical procedure. Shown on the drive unit 12 are a plurality of removable panels 16a-16n about and along the drive unit 12 enclosing the internal structure of the drive unit 12. An illustrative drive unit 12 is described in commonly assigned U.S. Pat. No. 7,935,077, titled THROMBECTOMY CATHETER DEPLOYMENT SYSTEM, the disclosure of which is hereby incorporated by reference. Centrally located in the drive unit 12 and aligned to the lower region of the panel 16g may be automatically opening doors 18 and 20 which open to expose the interior of the drive unit 12 to provide access to a carriage assembly 22. The carriage assembly 22, which may accommodate components of the pump/catheter assembly 14, as discussed further herein, is shown accessible via opening the closed doors 18 and 20. The drive unit 12 may include a catch basin for collecting fluid leakage from the components of the pump/catheter assembly 14. For example, a removable drip tray 24 is shown located on the front of the drive unit 12 extending from below the carriage assembly 22 toward the panel 16a. Other configurations of catch basins are also contemplated. The drip tray 24 and a removable receptacle 26 may collectively support and accommodate an effluent collection bag, such as effluent collection bag 28 of the pump/catheter assembly 14. In other instances, the drive unit 12 may include a different structure, such as a hook for hanging the effluent collection bag 28 from, or a shelf for setting the effluent collection bag 28 on. In instances where the carriage assembly 22 is movable, a carriage assembly activation switch 30 may be provided with the drive unit 12, such as located on panel 16g, to selectively position the carriage assembly 22 inwardly or outwardly. A user interface 32, including memory capabilities, may be provided with the drive unit 12, such as located at the upper region of the drive unit 12 between the upper regions of the upper side panels 16e and 16f. Saline bag hooks 34 and 36 may extend through the panels 16e and 16f to hang saline bags therefrom. The drive unit 12 may include a handle 42 as well as a plurality of wheels 52a-52n and brake pedals 54 for wheel lockage to assist in maneuvering the drive unit 12 by medical personnel.

The pump/catheter assembly 14, which may be a disposable single-use device, is shown unattached from the drive unit 12. The pump/catheter assembly 14 includes a pump 56 and a thrombectomy catheter 58. During use, a portion of the pump/catheter assembly 14 may be secured within a portion of the drive unit 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube 64 attached between the output of the pump 56 and the thrombectomy catheter 58 which may be coaxially arranged inside the effluent return tube 66, a catheter manifold 69 (between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the pump 56 and then to the thrombectomy catheter 58 through the high-pressure fluid supply tube 64.

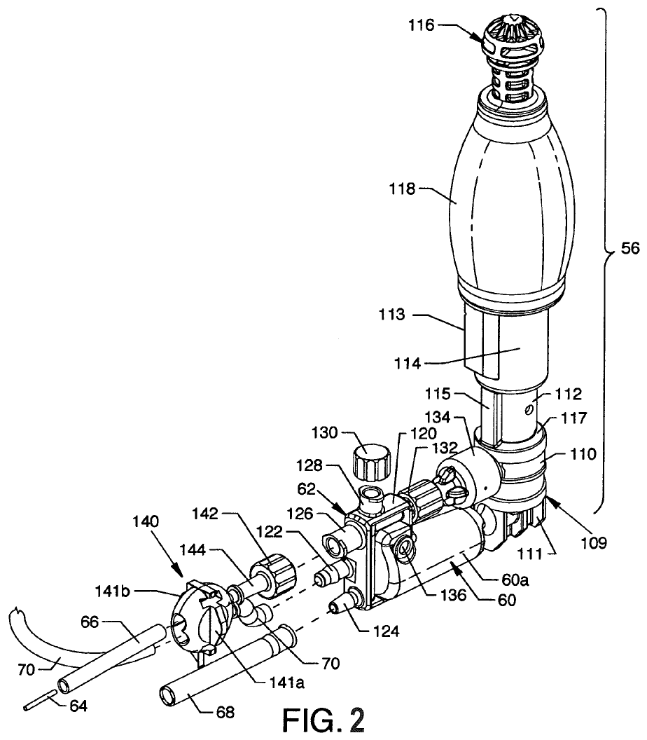

FIG. 2 is a partially exploded perspective view of several components of the pump/catheter assembly 14 generally including the pump 56, the bubble trap 60, the connection manifold assembly 62, and a fixture 140. The pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 to contain the pump 56 within the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example. A data plate 113 may also be included on the top body 114 for the inclusion of a barcode, an RFID tag, or other informational displays to determine operational parameters of the device.

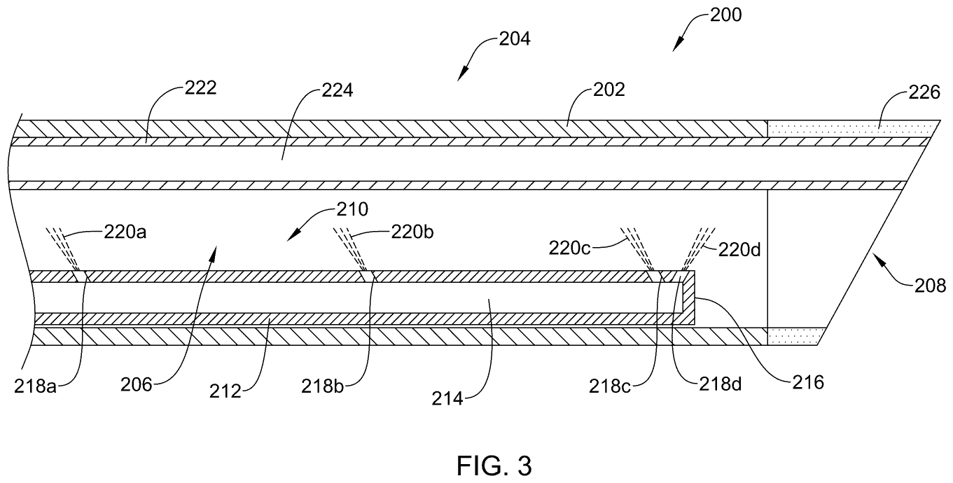

The pump 56 may include a hemispherically-shaped pump piston head 116 having a flexible boot 118 connected to and extending between the top body 114 and the pump piston head 116. In some instances, the geometrically configured lower portion 111 of the base 109 may serve as a mount for one end of the bubble trap 60 (FIG. 3).

The connection manifold assembly 62 may be secured directly to the other end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connecting extending between the connection manifold assembly 62 and the upper portion 110 of the base 109.

The bubble trap 60 may include mating halves of which one mating half 60a is shown. A hydrophobic filter 136 may be included at the upper forward region of the bubble trap half 60a. Another hydrophobic filter may be included on the second bubble trap half (not explicitly shown) which opposes the hydrophobic filter 136 on the bubble trap half 60a.

The fixture 140, and components associated therewith, assists in support and connection of the effluent return tube 66 to the effluent return port 126 by a connector 142 combined continuously with a connection tube 144, and also assists in support, passage and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting a roller pump (not explicitly shown) provided with the drive unit 12, such as located in the carriage assembly 22 or adjacent thereto.

FIG. 3 is a cross-sectional view of a distal end region 204 of another illustrative thrombectomy catheter 200. The thrombectomy catheter 200 may be one illustrative example of the thrombectomy catheter 58 described above. The thrombectomy catheter 200 may include a tubular member or catheter shaft 202 extending from a proximal end region (not explicitly shown) configured to remain outside the shaft to a distal end region 204. The catheter shaft 202 may be one illustrative example of, or be in fluid communication with, the effluent return tube 66 of the thrombectomy catheter 58 described above. A lumen 206 may extend from the proximal end region to the distal end region 204 of the catheter shaft 202. The catheter shaft 202 may terminate at a distally facing distal opening 208 at the distal end of the catheter shaft 202. In some instances, the distal opening 208 may be in a plane that extends generally orthogonal to a longitudinal axis of the catheter shaft 202. In other instances, the distal opening 208 may be in a plane that extends generally oblique to a longitudinal axis of the catheter shaft 202. In other words, in some examples, the distal end of the thrombectomy catheter 200 may be tapered relative to the longitudinal axis of the catheter shaft 202. Generally, the distal opening 208 may be an entrainment inflow orifice. While not explicitly shown, the catheter shaft 202 may include one or more markers (e.g., radiopaque marker bands) disposed along the catheter shaft 202. Further, while not explicitly shown, in some embodiments, the catheter shaft 202 may include one or more openings extending through a sidewall thereof, if desired.

The thrombectomy catheter/aspiration catheter 200 may further include a high-pressure fluid supply tube 210. The high-pressure fluid supply tube 210 may be one illustrative example of, or be in fluid communication with, the high-pressure fluid supply tube 66 of the thrombectomy catheter 58 described above. The high-pressure fluid supply tube 210 may be disposed within and extend through the lumen 206 of the catheter shaft 202. The high-pressure fluid supply tube 210 may include a supply tube wall 212 defining a lumen or fluid pathway 214 extending therethrough. In at least some instances, the high-pressure fluid supply tube 210 may have a closed distal end 216. Because of this, fluid may be able to pass distally through the fluid pathway 214 but does not exit the distal end. The high-pressure fluid supply tube 210 may extend along a length of the catheter shaft 202 with the distal end 216 located within the lumen 206 of the catheter shaft 202 proximal to the distal opening 208 at the distal end of the catheter shaft 202. A proximal end of the high-pressure fluid supply tube 210 may be in fluid communication with the pump 56 described herein, to provide high-pressure fluid to the fluid pathway 214 of the high-pressure fluid supply tube 210.

A plurality of jet orifices 218a-d (collectively, 218) may be defined along the supply tube wall 212. For example, the supply tube wall 212 may include two, three, four, five, six, or more jet orifices 218. The jet orifices 218 may be spaced along the supply tube wall 212 at any desired intervals. For example, each of the jet orifices 218 may be equidistantly spaced from adjacent jet orifices 218 along the length of the supply tube wall 212. In other instances, the jet orifices 218 may be arranged such that the spacing between adjacent jet orifices 218 near the distal end of the supply tube wall 212 is closer than the spacing between adjacent jet orifices 218 near the proximal end of the supply tube wall 212. For instance, the spacing between the orifices 218 may gradually increase as you move proximally along the length of the shaft, or the spacing may increase in a stepwise configuration. In some instances, some or all of the jet orifices 218 may be axially aligned along the supply tube wall 212. In other instances, one or more of the jet orifices 218 may be circumferentially offset from one another about the supply tube wall 212. A number of patterns are contemplated including a helical pattern, a pattern where no two jet orifices 218 are disposed at the same axial location, a regular pattern including two or more jet orifices 218 disposed at the same axial location, an irregular pattern (where some of the jet orifices 218 may or may not be disposed at the same axial location), etc.

The jet orifices 218 may be formed using a suitable method such as electron discharge machining, etching, cutting (e.g., including laser cutting), or the like. In some instances, one or more of the jet orifices 218 may have a substantially round shape. In other instances, one or more of the jet orifices 218 may have a substantially non-round shape (e.g., oval, polygonal, irregular, etc.). In some instances, the jet orifices 218 may be beveled or otherwise include a beveled surface. It is contemplated that a size and/or a shape of the jet orifices 218 may be varied to vary the velocity of the fluid exiting the jet orifices. For example, decreasing the size of the jet orifices 218 may increase the velocity of the fluid exiting the jet orifices 218. In some embodiments, the size of the jet orifices 218 may be varied based on the pressure capacity of the thrombectomy system, the number of jet orifices, the dimensions of the high-pressure fluid supply tube 210 (e.g., length, wall thickness, inner diameter, etc.), and/or combinations thereof. In some examples, the jet orifices 218 may have a cross-sectional dimension in the range of about 0.0018″ (0.0018 inches) to about 0.0022″. However, the jet orifices 218 can have a cross-sectional dimension of less than 0.0018″ or greater than 0.0022″, as desired.

Infusion of motive fluid through the lumen 214 of the supply tube wall 212 may result in fluid being jetted through the jet orifices 218 and the generation of a proximally directed aspiration force. At least some of the jet orifices 218a-c may be angled in a proximal direction or otherwise designed to infuse fluid (e.g., a motive fluid, a liquid, a gas or air, steam, a fluid with particles disposed therein, or the like) through the jet orifices 218a-c and into the lumen 206 of the catheter shaft 202 in a generally proximal direction as depicted by lines 220a-c representing motive jetted fluid projecting generally proximally from the jet orifices 218a-c. For example, each of the jet orifices 218a-c may be arranged at an acute angle to the longitudinal axis of the supply tube wall 212 such that the jet orifices 218a-c angle in a proximal direction. In some embodiments, one or more of the jet orifices 218d may be designed to infuse fluid (e.g., a motive fluid, a liquid, a gas or air, steam, a fluid with particles disposed therein, or the like) through the jet orifice(s) 218d and into the lumen 206 of the catheter shaft 202 in a generally distal direction as depicted by lines 220d representing motive jetted fluid projecting generally distally from the jet orifice 218d. For example, the jet orifice 218d may be arranged at an oblique angle to the longitudinal axis of the supply tube wall 212 such that the jet orifice 218d angles in a distal direction. It is contemplated that an angle of the jet orifices 218 and thus the motive jetted fluid 220 may be varied to adjust the velocity of the fluid exiting the jet orifices 218. As further described herein, the supply tube wall 212 may include one or more, or a plurality of proximally oriented or directed jet orifices 218a, 218b, 218c (i.e., jet orifices configured to direct fluid infused through the lumen 214 of the supply tube wall 212 in a proximal direction) and the supply tube wall 212 may include one or more, or a plurality of distally oriented or directed jet orifices 218d (i.e., jet orifices configured to direct fluid infused through the lumen 214 of the supply tube wall 212 in a distal direction). In some examples, the distally projecting jet orifice 218d may be axially aligned with one or more of the proximally projecting jet orifices 218a-c.

In other examples, the distally projecting jet orifice 218d may be circumferentially offset from one or more of the proximally projecting jet orifices 218a-c. For example, the distally projecting jet orifice 218d may be circumferentially offset from one or more of the proximally projecting jet orifices 218a-c by in the range of about 10° to about 350° or about 45° to about 135°.

The distally projecting jet orifice 218d may be the distalmost jet orifice, with the proximally projecting jet orifices 218a-c positioned proximal of the distally projecting jet orifice 218d. However, this is not required. In some embodiments, the distally projecting jet orifice 218d may be positioned proximal to at least one proximally projecting jet orifice 218a-c. While the supply tube wall 212 is illustrated as including only a single distally projecting jet orifice 218d, the supply tube wall 212 may include more than one distally projecting jet orifice, as desired. When more than one distally projecting jet orifice 218d is provided, the distally projecting jet orifices may be positioned at differing axial and/or circumferential locations from one another or similar axial and/or circumferential locations as one another, as desired. The distally projecting jet orifice(s) 218d may break up particles as they are drawn into the lumen 206 of the catheter shaft 202 while the proximally projecting jet orifices 218a-c may move particles proximally along the catheter shaft 202.

The performance of the thrombectomy catheter 200 and the high-pressure fluid supply tube 210 may be directly related to the velocity of the motive jetted fluid 220 exiting the jet orifices 218 and the shear-induced turbulent flux created by the jetted motive fluid 220. For example, the more powerful the jetted motive fluid 220, the higher the aspiration rates may be. It is further contemplated that the performance of the jet-powered aspiration catheter 200 may be directly related to the speed at which the clot can be entrained into the catheter 200, macerated, and removed from the shaft. Any clogging that occurs within the catheter shaft 202 may reduce or completely stop the removal of the clot. The addition of the distally projecting jet orifice 218d may macerate any clot that enters the distal opening 208 of the catheter shaft 202 thus helping prevent clogging. For example, at the point of impingement of the distally oriented motive jetted fluid 220d the motive jetted fluid 220d may deflect distally creating flow out the tip of the distal opening 208 of the catheter shaft 202, effectively macerating any clot that enters the tip of the device and eliminating or reducing risk of the distal opening 208 of the catheter shaft 202 becoming blocked or clogged. It is contemplated that the properties (size, shape, angle, number, spacing, etc.) of the jet orifices 218 may be varied to obtain a fluid velocity that creates an optimum de-clogging effect without hindering the proximal flow of a clot within the lumen 206 of the catheter shaft 202 or the clot evacuation rate.

The distally projecting jet orifice 218d may be proximally spaced a distance from the distal opening 208 of the catheter shaft 202. It is contemplated that the longitudinal location of the distally projecting jet orifice 218d on the supply tube wall 212 and relative to the distal opening 208 of the catheter shaft 202 may be varied based on a size of the aperture of the distally projecting jet orifice 218d, the velocity of the fluid within the lumen 214 of the supply tube wall 212, the angle of the distally projecting jet orifice 218d, or combinations thereof, etc. to ensure the distally oriented motive jetted fluid 220d impinges the inner surface of the catheter shaft 202. In one illustrative example, the distally projecting jet orifice 218d may be positioned such that the distally oriented motive jetted fluid 220d impinges an inner surface of the catheter shaft 202 such that the distally oriented motive jetted fluid 220d does not damage the vessel. For example, the distally projecting jet orifice 218d may be positioned such that the distally oriented motive jetted fluid 220d impinges an inner surface of the catheter shaft 202 in the range of about 0.070″ to about 0.090″ proximal to the distal end of the catheter shaft 202. This is just one example. The impingement location of the motive jetted fluid 220d of the distally projecting jet orifice 218d may be less than 0.070″ or more than 0.090″ proximal to the distal end of the catheter shaft 202, as desired.

In some instances, the jet orifices 218 may be oriented at an angle relative to the longitudinal axis of the supply tube wall 212. For example, the proximally projecting jet orifices 218a-c may be oriented at an oblique (e.g., acute) angle relative to the longitudinal axis of the supply tube wall 212 and/or oriented at an angle greater than zero degrees and less than ninety degrees relative to the longitudinal axis of the supply tube wall 212. It is contemplated that a distally projecting jet orifice 218d may be oriented at an oblique (e.g., obtuse) angle relative to the longitudinal axis of the supply tube wall 212 and/or oriented at an angle greater than 90 degrees and less than 180 degrees relative to the longitudinal axis of the supply tube wall 212. In other instances, the jet orifices 218 may be oriented perpendicular to the longitudinal axis of the supply tube wall 212 (e.g., at an angle of about 90 degrees relative to the longitudinal axis of the supply tube wall 212). The angle may or may not be the same for all the jet orifices 218.

In at least some instances, the jet orifices 218 may be understood as being arranged in series. In other words, the jet orifices 218 may be arranged such that adjacent jet orifices 218 are spaced longitudinally apart at various locations along the longitudinal axis of the supply tube wall 212. For example, the jet orifices 218 may be uniformly or non-uniformly spaced along a length of the supply tube wall 212. This may position the jet orifices 218 at axially spaced apart locations within the catheter shaft 202 and along the length thereof. For example, the jet orifices 218 may be spaced along an entire length of the supply tube wall 212 and correspondingly along an entire length of the catheter shaft 202, or portions thereof, as desired. In some examples, the jet orifices 218 may be spaced at intervals in the range of every 5 inches to every 15, or in the range of every 6 inches to every 12 inches along a length of the supply tube wall 212. In other instances, the spacing between the jet orifices 218 may be less than every 5 inches or greater than every 15 inches. Accordingly, motive fluid leaves via the jet orifices 218 forming a jetted motive fluid 220a-d (collectively, 420). This jetted motive fluid 220 enters an entrainment material where the shear layer between the two causes turbulence, mixing, and transfer of momentum. Entrainment material may enter the distal opening 208 and then may be urged proximally by momentum transfer. As the mixture of jetted motive fluid 220 and entrainment material migrates proximally, the material may sequentially approach a number of jet orifices 218. Upon interaction with the jetted motive fluid 220 from each individual jet orifice 218, the momentum in the entrainment material mixture may increase, and the thrombogenic material may more readily flow proximally through the catheter shaft 202 for removal. The increase in momentum may allow for the catheter shaft 202 to be used without a second or outflow orifice (e.g., positioned proximally of the distal opening 208). Alternatively, some of the entrapped thrombogenic material may exit the catheter shaft 202 through a second orifice (not shown), e.g., in a sidewall of the catheter shaft 202, positioned proximal to the distal opening 208, recirculate to the distal opening 208 (e.g., one or more times), and then move proximally through the lumen 206 of the catheter shaft 202.

It is further contemplated that the distally oriented motive jetted fluid 220d may be partially to fully entrained by the force generated by the proximally oriented motive jetted fluid 220a-c. When the clot/thrombus reaches the distally oriented motive jetted fluid 220d, the shear stress may masticate the clot/thrombus. It is contemplated that when the distal opening 208 of the catheter shaft 202 is sealed with a clot/thrombus, the force generated by the proximally oriented motive jetted fluid 220a-c may be transferred to the surface of the clot/thrombus in a proximal direction. As a result, the distally oriented motive jetted fluid 220d may no longer be entrained and may transfer force in the distal direction to the surface of the clot/thrombus. Thus, when the distal opening 208 is clogged or plugged, an extreme shear mechanism of action is created where the distal and proximal force vectors combine together to focus all of the shear stress to the surface of the clot/thrombus to masticate the clot/thrombus and unplug the distal opening 208. It is contemplated that the shear stress on the clot/thrombus may be much larger in magnitude when the distally oriented motive jetted fluid 220d is at a smaller angle (e.g., closer to 180 degrees relative to the longitudinal axis of the supply tube wall 212 than to orthogonal to the longitudinal axis of the supply tube wall 212).

FIG. 3 illustrates that the thrombectomy catheter 200 may further include a tip member 226 positioned along the distal end region 204. Additionally, FIG. 3 illustrates that the thrombectomy catheter 200 may also include a guidewire shaft 222. The guidewire shaft 222 may extend within the lumen 206 of the catheter shaft 202. In some examples, a proximal end of the guidewire shaft 222 may be coupled (e.g., attached, bonded) to the catheter manifold 69 (shown in FIG. 1). Additionally, as will be discussed in greater detail herein, the distal end region of the guidewire shaft 222 may be bonded to the tip member 226. It can be appreciated that, in some examples, the portion of the guidewire shaft 222 extending between the catheter manifold 69 and the tip member 226 may remain attached to the catheter shaft 202. For example, a proximal portion of the guidewire shaft 222 may be attached to the catheter manifold 69, a distal portion of the guidewire shaft 222 may be attached to the tip member 226 and the portion of the guidewire shaft 222 extending between the catheter manifold 69 and the tip member 226 may be unattached and free to move within the lumen 206 of the catheter shaft 202. Further, FIG. 3 illustrates that the guidewire shaft 222 may include a lumen 224 configured to permit a guidewire to extend within.

It can be appreciated that the catheter shaft 202 may be formed from a polymer material. For example, the catheter shaft 202 may be formed from a polymer material including, but not limited to a thermoplastic polymer (e.g., Pebax®). Other suitable polymers which may be utilized to form the tip member 226 may include Vestamid®, Grilamid®, polyamides including Nylon 6, Nylon 66, Nylon 11, Nylon 12, polyether block amide copolymer including 32D Pebax®, 35D Pebax®, 48D Pebax®, 55D Pebax®, 68D Pebax®, 72D Pebax®, Pebax® MED, Rilsan® MED, Rilsamid® MED, Rilsan® Clear MED and Kynar® MED. A non-limiting list of examples which may be utilized to form the catheter shaft 202 is disclosed below.

The guidewire shaft 222 may be formed from a polymer material including, but not limited to a polyimide. Other suitable polymers which may be utilized to form the guidewire shaft 222 may include PEEK (polyether ether ketone). A non-limiting list of examples which may be utilized to form the guidewire shaft 222 is disclosed below.

It can be appreciated that constructing the guidewire shaft 222 from a thermoset polyimide provides sufficient strength to the guidewire shaft 222, thereby allowing the guidewire shaft 222 to withstand forces of the jetted motive fluid 220a-d leaving the jet orifices 218a-d. However, it can be further appreciated that the thermoset polyimide material utilized to form the guidewire shaft 222 may not be capable of forming a chemical bond to the thermoplastic polymer used to form the catheter shaft 202 and the tip member 226. Accordingly, it may be desirable to design a portion of the guidewire shaft 222 to include one or more features which permit the thermoset polyimide material utilized to form the guidewire shaft 222 to form a mechanical bond to the thermoplastic polymer used to form the tip member 226.

The materials that can be used for the various components of the catheter 200 may include those commonly associated with medical devices. However, this is not intended to limit the devices and methods described herein, as the discussion may be applied to other components, devices, or systems disclosed herein.

The components of the catheter 200 (and/or other systems disclosed herein) may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the components of the catheter 200 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the components of the catheter 200 (and/or other systems disclosed herein) in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the components of the system 10 (and/or other systems disclosed herein) to achieve the same result.

In some embodiments, a degree of Magnetic Resonance Imaging (MRI) compatibility is imparted into the catheter 200 (and/or other systems disclosed herein). For example, components of the catheter 200 (and/or other systems disclosed herein), may be made of a material that does not substantially distort the image and create substantial artifacts (e.g., gaps in the image). Certain ferromagnetic materials, for example, may not be suitable because they may create artifacts in an MRI image. The components of the catheter 200 (and/or other systems disclosed herein) or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

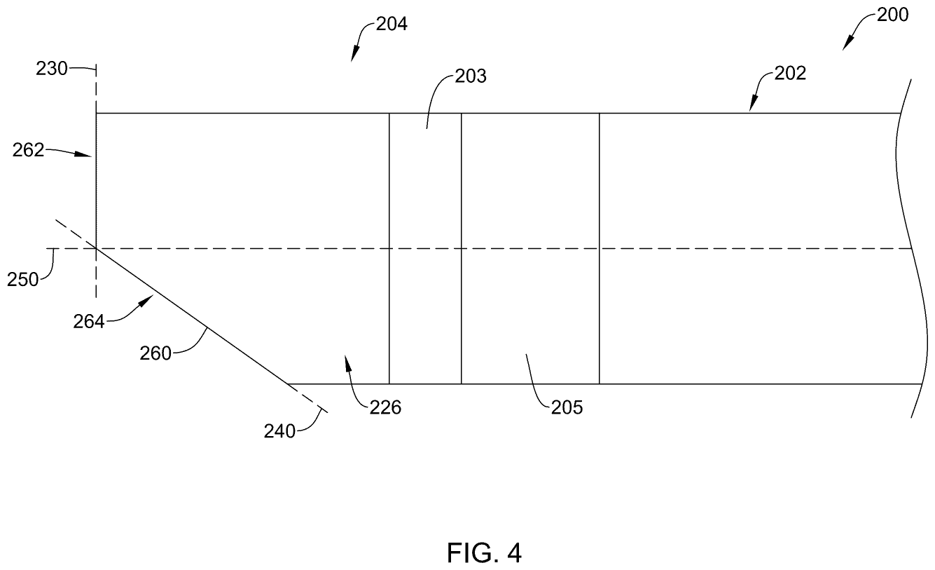

FIG. 4 illustrates a side view of an alternative configuration of the distal end region 204 of catheter 200. Catheter 200 may further include catheter shaft 202 which includes or is coupled to a distal tip member 226. A radiopaque marker band 203 may be disposed about the outer circumferential surface of the catheter shaft 202 and located near the distal tip member 226 or near the catheter 200 distal end region 204. One or more radiopaque marker bands 203 may be present and may alternatively take the form of a radiopaque coating, a radiopaque section, a strip of radiopaque material, a strip of radiopaque material comprising indicia and/or a pattern (i.e., a helical pattern, a zig-zag pattern, a serpentine pattern, an undulating pattern, a sinusoidal pattern, or the like). The catheter shaft 202 of the catheter 200 may further include a saddle region 205. Saddle region 205 may be designed to add structural integrity to the catheter shaft 202 of catheter 200 such that the catheter 200 may resist deformation and damage under high fluid forces and fluid pressures. The saddle region 205 may be formed partially or entirely of stainless steel, or may be formed partially or entirely by materials including but not limited to: a metallic material, such as nitinol, a plastic material, such as polyether ether ketone (PEEK), or the like.

The distal tip member 226 shown in FIG. 4 and in other examples may include a distal facing outer surface 260 circumferentially surrounding the distal opening of the distal tip member 226. As shown in FIG. 4, the distal outer surface 260 (i.e., distally facing outer surface) of the distal tip member 226 (i.e., the rim or outer edge of the distal tip member 226 which faces distally) may reside in more than one plane. For instance, a first portion 262 of the distal outer surface 260 of the distal tip member 226 may reside in a first plane 230, while a second portion 264 of the distal outer surface 260 of the distal tip member 226 may reside in a second plane 240 that is non-parallel to and intersects with the first plane 230. The first plane 230 may extend transverse (i.e., non-parallel to) to the central longitudinal axis 250 of the distal tip member 226, and in some instances, the first plane 230 may be a plane that extends orthogonal to the central longitudinal axis 250 of the distal tip member 226. In other words, the first plane 230 may be set at an angle of about 90 degrees or exactly 90 degrees with respect to the central longitudinal axis 250 of the distal tip member 226, effectively extending perpendicular to the central longitudinal axis 250 of the distal tip member 226. Other angles and ranges of angles for the first plane 230 are also contemplated. In some instances, the first plane 230 may be set at an angle in the range of 75 degrees to 90 degrees or in a range of 60 degrees to 90 degrees to the central longitudinal axis 250 of the distal tip member 226. Alternatively or additionally to any of the examples disclosed herein, the first plane 230 may ascribe to an angle of between 75 degrees and 95 degrees with respect to the central longitudinal axis 250 of the distal tip member 226. In other non-limiting examples, the first plane 230 may ascribe to an angle of between 55 degrees and 125 degrees with respect to the central longitudinal axis 250 of the distal tip member 226. Other angles and ranges of angles within the disclosed ranges may be implemented as desired.

The second plane 240 may extend at an acute angle to the central longitudinal axis 250 of the distal tip member 226. For example, in some instances, the second plane 240 may ascribe to an acute angle of between 5 degrees and 35 degrees with respect to the central longitudinal axis 250 of the distal tip member 226. In other non-limiting examples, the second plane 240 may ascribe to an angle of between 20 degrees and 35 degrees with respect to the central longitudinal axis 250 of the distal tip member 226. In yet other non-limiting examples, the second plane 240 may ascribe to an angle of between 25 degrees and 60 degrees with respect to the central longitudinal axis 250 of the distal tip member 226. Other angles and ranges of angles known in the art may be implemented as desired.

As noted above, the first plane 230 may intersect the second plane 240, and thus the first portion 262 of the distal outer surface 260 of the distal tip member 226 which lies in the first plane 230 may be non-parallel to the second portion 264 of the distal outer surface 260 of the distal tip member 226 which lies in the second plane 240. For instance, the first plane 230 may ascribe to an angle or a range of angles relative to the second plane 240 while intersecting the second plane 240. The first plane 230 may be set at an angle of about 5 degrees, an angle of about 10 degrees, an angle of about 15 degrees, an angle of about 20 degrees, an angle of about 25 degrees, an angle of about 30 degrees, an angle of about 35 degrees, an angle of about 40 degrees, an angle of about 45 degrees, an angle of about 50 degrees, an angle of about 55 degrees, an angle of about 60 degrees, an angle of about 65 degrees, an angle of about 70 degrees, an angle of about 75 degrees, an angle of about 80 degrees, an angle of about 85 degrees, an angle of about 90 degrees, an angle of about 95 degrees, an angle of about 100 degrees, an angle of about 105 degrees, an angle of about 110 degrees, an angle of about 115 degrees, an angle of about 120 degrees, an angle of about 125 degrees or an angle of about 130 degrees or more with respect to the second plane 240.

As a first portion 262 of the distal outer surface 260 of the distal tip member 226 may reside in the first plane 230, at least a second portion 264 of the distal outer surface 260 of the distal tip member 226 may reside in the second plane 240, different than the first plane 230. The first portion 262 of the distal outer surface 260 that resides in the first plane 230 may be any desired portion of the circumference of the distal outer surface 260.

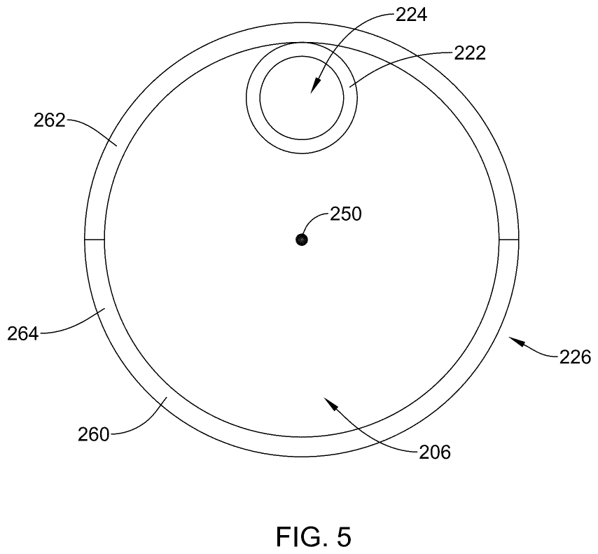

FIG. 5 shows a distal end view of the distal tip member 226 of FIG. 4 showing the distal facing outer surface 260 extending around the circumference of the distal tip member 226. The distal outer surface 260 may have a width (measured in the radial direction from the central longitudinal axis) equivalent to the wall thickness of the distal tip member 226. The distal outer surface 260 of the distal tip member 226 may include a first portion 262 that lies in the first plane 230 and a second portion 264 that lies in the second plane 240. The first plane 230 and the second plane 240 may intersect at a point within the distal opening of the distal tip member 226.

As noted above, the first portion 262 of the distal outer surface 260 that resides in the first plane 230 may be any desired portion of the circumference of the distal outer surface 260. For example, in some instances the first portion 262 of the distal outer surface 260 that resides in the first plane 230 may be extend around about 180 degrees of the circumference of the distal outer surface 260 (i.e., in instances in which the first plane 230 intersects the second plane 240 at the central longitudinal axis 250). In other instances, the first portion 262 of the distal outer surface 260 that resides in the first plane 230 may be extend around less than 180 degrees of the circumference of the distal outer surface 260, such as 170 degrees or less, 150 degrees or less, or 120 degrees or less, for example (i.e., in instances in which the first plane 230 intersects the second plane 240 at a point distal of the point the second plane 240 intersects the central longitudinal axis 250). In yet other instances, the first portion 262 of the distal outer surface 260 that resides in the first plane 230 may be extend around more than 180 degrees of the circumference of the distal outer surface 260, such as 190 degrees or more, 210 degrees or more, or 240 degrees or more, for example (i.e., in instances in which the first plane 230 intersects the second plane 240 at a point proximal of the point the second plane 240 intersects the central longitudinal axis 250).

Likewise, in some instances the second portion 264 of the distal outer surface 260 that resides in the second plane 240 may be extend around about 180 degrees of the circumference of the distal outer surface 260 (i.e., in instances in which the first plane 230 intersects the second plane 240 at the central longitudinal axis 250). In other instances, the second portion 264 of the distal outer surface 260 that resides in the second plane 240 may be extend around more than 180 degrees of the circumference of the distal outer surface 260, such as 190 degrees or more, 210 degrees or more, or 240 degrees or more, for example (i.e., in instances in which the first plane 230 intersects the second plane 240 at a point distal of the point the second plane 240 intersects the central longitudinal axis 250). In yet other instances, the second portion 264 of the distal outer surface 260 that resides in the second plane 240 may be extend around less than 180 degrees of the circumference of the distal outer surface 260, such as 170 degrees or less, 150 degrees or less, or 120 degrees or less, for example (i.e., in instances in which the first plane 230 intersects the second plane 240 at a point proximal of the point the second plane 240 intersects the central longitudinal axis 250).

One or more of the first portion 262 of the distal outer surface 260 lying in the first plane 230 and second portion 264 of the distal outer surface 260 lying in the second plane 240 may extend proximally from the distalmost end of the distal tip member 226 of catheter 200. For example, the second portion of the distal outer surface 260 lying in the second plan 240 may extend proximally form the distalmost end of the distal tip member 226. It is also contemplated that only the second portion of the distal outer surface 260 lying in the second plan 240 may extend proximally form the distalmost end of the distal tip member 226 while the first portion of the distal outer surface 260 lying in the first plane 230 never passes proximally beyond a plane orthogonal to the central longitudinal axis 250 and to which the distalmost end of the distal tip member 226 extends to.

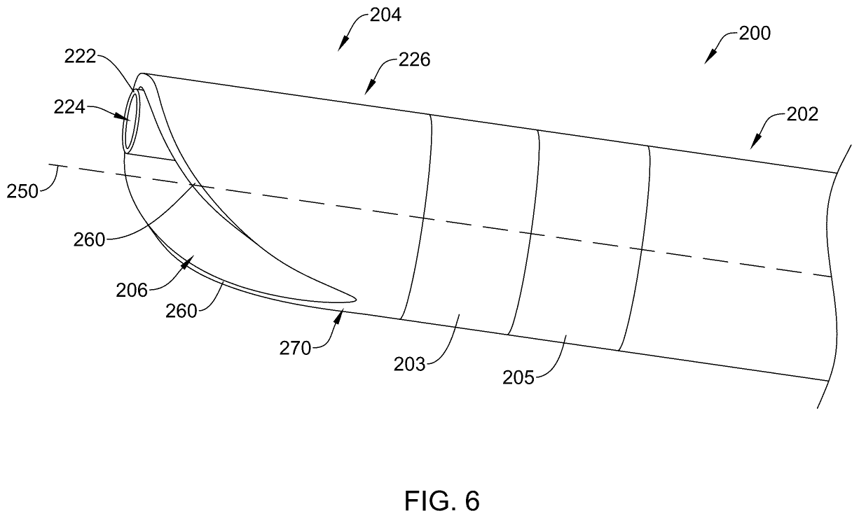

FIG. 6 shows a side view of another alternative configuration of the distal end region 204 of catheter 200. Catheter 200 may further include catheter shaft 202 which includes or is coupled to distal tip member 226. A radiopaque marker band 203 may be disposed about the outer circumferential surface of the catheter shaft 202 and located near the distal tip member 226 or near the catheter 200 distal end region 204. One or more radiopaque marker bands 203 may be present and may alternatively take the form of a radiopaque coating, a radiopaque section, a strip of radiopaque material, a strip of radiopaque material comprising indicia and/or a pattern (i.e., a helical pattern, a zig-zag pattern, a serpentine pattern, an undulating pattern, a sinusoidal pattern, or the like).

The catheter shaft 202 of the catheter 200 may further include a saddle region 205. Saddle region 205 may be designed to improve the strength of the catheter shaft 202 of catheter 200 such that the catheter 200 may resist deformation or damage under high forces and high pressures. The saddle region 205 may be formed partially or entirely of stainless steel, or may be formed partially or entirely by materials including but not limited to a metallic material, such as nitinol, a plastic material, such as polyether ether ketone (PEEK), or the like.

The distal tip member 226 shown in FIG. 6 and in other examples may include a distal facing outer surface 260 circumferentially surrounding the distal opening of the distal tip member 226. When viewed from the side (i.e., perpendicular to the central longitudinal axis of the distal tip member 226), as in FIG. 6, the distal facing outer surface 260 may extend along an arcuate (i.e., convex) path that runs transverse to the central longitudinal axis 250 of the distal tip member 226. In other words, the rim, or edge of the distal tip member 226 that faces distally (synonymous with the distal facing outer surface 260) is formed such that the distal facing outer surface 260 conforms to a curved path that is arcuate (i.e., convex) when viewing the distal end 204 of the catheter 200 from a lateral direction. In some instances, the proximalmost portion of the distal facing outer surface 260 may extend generally parallel with the central longitudinal axis 250, whereas the distalmost portion of the distal facing outer surface 260 may extend generally or substantially perpendicular with the central longitudinal axis 250, with the distal facing outer surface 260 continuously curving therebetween. Other geometries and patterns for the path of the distal outer facing surface 260 are contemplated, including but not limited to the distal outer surface 260 following a parabolic path, an undulating path, a convex path, a path including multiple inflection points 280, or any equivalent or combinatory path or pattern which may be implemented as desired.

As the distal facing outer surface 260 follows a convex path, it is understood that the various portions of the distal facing outer surface 260 lie in different plane non-parallel to one another. The various planes are tangential to the distal facing outer surface 260 at various locations along the distal facing outer surface 260. For example, a first portion of the distal facing outer surface 260 would lie in a first plane tangential to the distal facing outer surface 260 at a first point on the distally facing outer surface 260, and a second portion of the distal facing outer surface 260 would lie in a second plane tangential to the distal facing outer surface 260 at second point on the distal facing outer surface 260 distal of the first point, a third portion of the distal facing outer surface 260 would lie in a third plane tangential to the distal facing outer surface 260 at a third point on the distally facing outer surface 260 distal of the second point, and so on. Said another way, the distal facing outer surface 260 may have a continuously varying slope as the distally facing outer surface extends distally.

The distal facing outer surface 260 may terminate at its proximalmost extent in a vertex 270 as shown in FIG. 6. Vertex 270 may be rounded, square, terminate at a point, pass through an inflection point 280, and/or pass through a concave inflection point 280. As the distal facing outer surface 260 traces a path of geometry, it may further include multiple inflection points. In other words, the path of the distal facing outer surface 260 may inflect, or change direction multiple times as it transcribes around the central longitudinal axis 250 of the distal tip member 226 as the distal outer surface 260 extends around the perimeter (e.g., circumference) of the distal tip member 226. As shown in at least FIG. 6, the vertex 270 may serve as an inflection point of the distal outer facing surface 260 as the path of the distal outer facing surface 260 changes direction through the valley (i.e. lowest point, lowest concavity) of the vertex 270 of the distal tip member 226. In this and other examples, one or more additional inflection points may be formed in the distal outer facing surface 260 of the distal tip member 226. For instance, an inflection point may be present at the distalmost end of the distal tip member 226 (i.e., the end farthest away from the user of the device). This inflection point is shown in at least FIG. 6 as the path of the distal outer surface 260 of the distal tip member 226 changes direction as it transcribes an arc around the guidewire lumen 224. The inflection point at the distalmost extent of the distal tip member 226 may be circumferentially opposite (i.e., 180° away from) the vertex 270, in some instances. In this and other examples, additional inflection points are contemplated and may be present on any other portion of the distal outer surface 260 of the distal tip member 226. In further non-limiting examples, the distal outer surface 260 of the distal tip member 226 may include at least one inflection point, at least two inflection points, at least three inflection points, at least four inflection points, or at least five inflection points or more.

The vertex 270 of this and other examples may be positioned such that the nadir (i.e. lowest point of vertex 270) is located near the distal end of the catheter shaft 202. In this and other examples, the lowest point, or nadir, of the vertex 270 may be located about 0.1 mm from the distal end of the catheter shaft 202. In other non-limiting examples, the nadir of the vertex 270 may be located about 0.15 mm, about 0.2 mm, about 0.25 mm, about 0.3 mm, about 0.35 mm, about 0.4 mm, about 0.5 mm, about 0.75 mm, about 1 mm, about 1.25 mm, about 1.5 mm, about 1.75 mm, about 2 mm, about 2.5 mm, about 3 mm, about 4 mm or about 5 mm or more from the distal end of the catheter shaft 202. The vertex 270 of this and other examples may be positioned such that the nadir (i.e. lowest point of vertex 270) is located near at the proximalmost extent of the distal opening of the distal tip member 226.

As shown in FIG. 6 and other examples, the distal opening 206 of distal tip member 226 may be formed such that it creates a vertex 270 in which the gap between walls of the distal opening 206 of the distal tip member 226 converge to form a vertex 270 located a distance proximal of the distalmost end of the distal tip member 226. In other words, the vertex 270 may be formed such that its nadir (i.e. lowest point) lies at a distance proximal of about 0.25 mm from the distalmost end of the distal tip member 226. In other non-limiting examples, the vertex 270 may lie at a distance of about 0.35 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 5.25 mm, about 5.5 mm, about 5.75 mm or about 6 mm or more proximal of the distalmost end of the distal tip member 226.

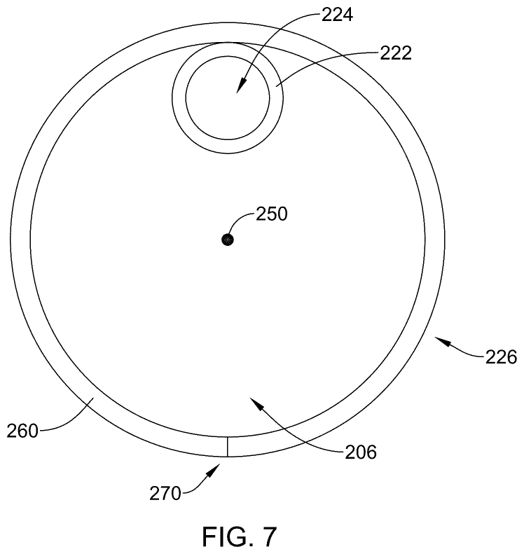

FIG. 7 shows a distal end view of the distal tip member 226 of FIG. 6 showing distal outer surface 260 extending around the circumference of the distal tip member 226. The distal outer surface 260 may have a width (measured in the radial direction from the central longitudinal axis) equivalent to the wall thickness of the distal tip member 226. The distal outer surface 260 may be formed such that it conforms to a convex path that transcribes around the central longitudinal axis 250 of the distal tip member 226.

It is further contemplated that the distal outer surface 260 of the distal tip member 226 may include one or more inflection points. In other non-limiting examples, the distal outer surface 260 of the distal tip member 226 may include two or more inflection points, three or more inflection points, four or more inflection points, or five or more inflection points. Alternatively or additionally, the inflection points may inflect abruptly (i.e. turn or change direction at an angle or arc greater than 30 degrees) or may inflect gradually (i.e. turn or change direction at an angle or arc less than 30 degrees).

The distal tip member 226 of this and other examples may further accommodate a guidewire tube 222 extending within the distal tip member 226 and which defines a guidewire lumen 224 for passage of a guidewire therein and therethrough. The guidewire tube 222 may be offset from the central longitudinal axis of the distal tip member 226, and in some instances, the guidewire tube 222 may be attached to or near an interior wall of the catheter shaft 202 and the distal tip member 226. In some instances, the guidewire tube 222 may be engaged with and/or secured to the inner wall of the distal tip member 226 circumferentially opposite (i.e., 180° away from) from the vertex 270, in some instances.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An aspiration catheter, comprising:
a catheter shaft having a proximal end region, a distal end region and a lumen extending therein;
a distal tip member having a length, a central longitudinal axis, a lumen extending therein and a distal opening, wherein the distal tip member is coupled to the distal end region of the catheter shaft;
a guidewire shaft having a proximal end region and a distal end region;
wherein the guidewire shaft extends within at least a portion of the lumen of the catheter shaft and the lumen of the distal tip member;
wherein the distal tip member comprises a distal facing outer surface surrounding the distal opening of the distal tip member; and
wherein a first portion of the distal facing outer surface of the distal tip member lies in a first plane, and a second portion of the distal facing outer surface of the tip member lies in a second plane, different from the first plane.

2. The aspiration catheter of claim 1, wherein the first plane is substantially orthogonal to the central longitudinal axis.

3. The aspiration catheter of claim 1, wherein the second plane intersects the first plane at a point distal of a proximal edge of the distal opening of the distal tip member.

4. The aspiration catheter of claim 1, wherein the second plane intersects the first plane at an angle ranging between 15 and 60 degrees with respect to the first plane.

5. The aspiration catheter of claim 1, wherein the second plane intersects the central longitudinal axis at an angle ranging from 25 degrees to 60 degrees.

6. The aspiration catheter of claim 1, wherein the guidewire shaft extends to the distally facing outer surface.

7. The aspiration catheter of claim 1, wherein the first portion extends about 180 degrees about a circumference of the distal facing outer surface and the second portion extends about 180 degrees about the circumference of the distal facing outer surface.

8. The aspiration catheter of claim 1, wherein the first portion extends less than 180 degrees about a circumference of the distal facing outer surface and the second portion extends greater than 180 degrees about the circumference of the distal facing outer surface.

9. The aspiration catheter of claim 1, wherein the first portion extends greater than 180 degrees about a circumference of the distal facing outer surface and the second portion extends less than 180 degrees about the circumference of the distal facing outer surface.

10. The aspiration catheter of claim 1, wherein the distal facing outer surface follows a convex pathway when viewed laterally.

11. The aspiration catheter of claim 10, wherein the first plane and the second plane are both tangent to the convex pathway.

12. The aspiration catheter of claim 10, wherein the distal facing outer surface includes a vertex at a proximalmost extent of the distal facing outer surface.

13. The aspiration catheter of claim 12, wherein the guidewire shaft is positioned along an inner surface of the distal tip member opposite the vertex.

14. The aspiration catheter of claim 10, wherein the convex pathway of the distal facing outer surface of the tip member is substantially perpendicular to the central longitudinal axis at a distalmost extent of the distal facing outer surface.

15. The aspiration catheter of claim 14, wherein the distal facing outer surface continuously curves from a vertex at a proximalmost extent of the distal facing outer surface to the distalmost extent of the distal facing outer surface.

16. An aspiration catheter, comprising:
a catheter shaft having a proximal end region, a distal end region and a lumen extending therein;
a distal tip member having a length, a central longitudinal axis, a lumen extending therein and a distal opening, wherein the distal tip member is coupled to the distal end region of the catheter shaft;
a guidewire shaft having a proximal end region and a distal end region;
wherein the guidewire shaft extends within at least a portion of the lumen of the catheter shaft and the lumen of the distal tip member;
wherein the distal tip member comprises a distal facing outer surface surrounding the distal opening of the distal tip member; and
wherein a first portion of the distal facing outer surface of the distal tip member lies in a first plane perpendicular to the central longitudinal axis, and a second portion of the distal facing outer surface of the tip member extends proximally from the first portion of the distal facing outer surface, the second portion lying in a second plane at an acute angle to the central longitudinal axis.

17. The aspiration catheter of claim 16, wherein the first portion extends about 180 degrees about a circumference of the distal facing outer surface and the second portion extends about 180 degrees about the circumference of the distal facing outer surface.

18. The aspiration catheter of claim 16, wherein the first portion extends less than 180 degrees about a circumference of the distal facing outer surface and the second portion extends greater than 180 degrees about the circumference of the distal facing outer surface.

19. An aspiration catheter, comprising:
a catheter shaft having a proximal end region, a distal end region and a lumen extending therein;
a distal tip member having a length, a central longitudinal axis, a lumen extending therein and a distal opening, wherein the distal tip member is coupled to the distal end region of the catheter shaft;
a guidewire shaft having a proximal end region and a distal end region;
wherein the guidewire shaft extends within at least a portion of the lumen of the catheter shaft and the lumen of the distal tip member;
wherein the distal tip member comprises a distal facing outer surface surrounding the distal opening of the distal tip member; and
wherein the distal facing outer surface follows a convex pathway when viewed from a side of the catheter shaft.

20. The aspiration catheter of claim 19, wherein the distal facing outer surface continuously curves from a vertex at a proximalmost extent of the distal facing outer surface to a distalmost extent of the distal facing outer surface, wherein the convex pathway of the distal facing outer surface of the tip member is substantially perpendicular to the central longitudinal axis at the distalmost extent of the distal facing outer surface.
